(19) United States
(12) Patent Application Publication
(10) Pub. No.: US 2024/0185094 A1
(43) Pub. Date: Jun. 6, 2024

(54) PREDICTION MODEL GENERATION APPARATUS, PREDICTION MODEL GENERATION METHOD, AND NON-TRANSITORY COMPUTER READABLE MEDIUM

(71) Applicant: NEC Corporation, Minato-ku, Tokyo (JP)

(72) Inventors: Kenji ARAKI, Tokyo (JP); Kosuke NISHIHARA, Tokyo (JP); Yuki KOSAKA, Tokyo (JP)

(73) Assignee: NEC Corporation, Minato-ku, Tokyo (JP)

(21) Appl. No.: 18/285,307

(22) PCT Filed: Apr. 9, 2021
(86) PCT No.: PCT/JP2021/015089

§ 371 Date: Oct. 2, 2023

(51) Int. Cl. G06N 5/022; G06N 7/01

(52) U.S. Cl. CPC G06N 5/022; G06N 7/01

(57) ABSTRACT

A prediction model generation apparatus according to an example embodiment of the present disclosure includes: at least one memory storing instructions; and at least one processor configured to execute the instructions to: divide a region in which a probability distribution of an objective variable exists into a plurality of small regions according to a property of the objective variable for learning data including the objective variable; model an existence probability that the objective variable belongs to each of the small regions; use the learning data to model, for each of the small regions, a probability distribution related to a possible value of the objective variable in the small region under a condition that the objective variable belongs to the small region; and constructs a prediction model of the objective variable by integrating the modeled probability distribution for each of the small regions using the existence probability.

### TECHNICAL FIELD

The present disclosure relates to a prediction model generation apparatus, a prediction model generation method, and a non-transitory computer readable medium.

### BACKGROUND ART

In recent years, there have been considered systems for predicting the degrees of recovery of patients from a disease when the patients are hospitalized. For example, Patent Literature 1 describes a technology with which a medical information processing system refers to an electronic medical record information group obtained from patients hospitalized in an acute phase medical facility, performs machine learning of a transfer destination of each patient from the acute phase medical facility, and predicts a transfer destination of a target patient based on a learning result.

### CITATION LIST

### Patent Literature

Patent Literature 1: International Patent Publication No. WO 2019/044620

### SUMMARY OF INVENTION

### Technical Problem

An object of this disclosure is to improve the technology disclosed in Patent Literature 1.

### Solution to Problem

A prediction model generation apparatus according to one aspect of the present example embodiment includes: a division means for dividing a region in which a probability distribution of an objective variable exists into a plurality of small regions according to a property of the objective variable for learning data including the objective variable; an existence probability modeling means for modeling an existence probability that the objective variable belongs to each of the small regions; a probability distribution modeling means for using the learning data to model, for each of the small regions, a probability distribution related to a possible value of the objective variable in the small region under a condition that the objective variable belongs to the small region; and a model construction means for constructing a prediction model of the objective variable by integrating the modeled probability distribution for each of the small regions using the existence probability.

A prediction model generation method according to one aspect of the present example embodiment is that a prediction model generation apparatus performs: dividing a region in which a probability distribution of an objective variable exists into a plurality of small regions according to a property of the objective variable for learning data including the objective variable; modeling an existence probability that the objective variable belongs to each of the small regions; using the learning data to model, for each of the small regions, a probability distribution related to a possible value of the objective variable in the small region under a condition that the objective variable belongs to the small region; and constructing a prediction model of the objective variable by integrating the modeled probability distribution for each of the small regions using the existence probability.

A non-transitory computer-readable medium according to an aspect of the present example embodiment stores a program for causing a computer to execute: dividing a region in which a probability distribution of an objective variable exists into a plurality of small regions according to a property of the objective variable for learning data including the objective variable; modeling an existence probability that the objective variable belongs to each of the small regions; using the learning data to model, for each of the small regions, a probability distribution related to a possible value of the objective variable in the small region under a condition that the objective variable belongs to the small region; and constructing a prediction model of the objective variable by integrating the modeled probability distribution for each of the small regions using the existence probability.

### BRIEF DESCRIPTION OF DRAWINGS

FIG. 1 is a block diagram illustrating an example of a prediction model generation apparatus according to a first example embodiment.

FIG. 2 is a block diagram illustrating an example of a probability distribution modeling unit according to the first example embodiment.

FIG. 3 is a flowchart illustrating a processing example of the prediction model generation apparatus according to the first example embodiment.

FIG. 4 is a block diagram illustrating an example of a prediction system according to a second example embodiment.

FIG. 5A is a block diagram illustrating an example of a prediction model generation unit according to the second example embodiment.

FIG. 5B is a block diagram illustrating an example of a modeling unit according to the second example embodiment.

FIG. 6 is a block diagram illustrating an example of a prediction unit according to the second example embodiment.

FIG. 7 is a flowchart illustrating a processing example of the prediction system according to the second example embodiment.

FIG. 8A is a graph illustrating a probability distribution of a theoretical recovery degree.

FIG. 8B is a graph illustrating a probability distribution of an actual recovery degree.

FIG. 9 is a block diagram illustrating an example of a prediction system according to a third example embodiment.

FIG. 10A is a block diagram illustrating an example of a prediction model generation unit according to the third example embodiment.

FIG. 10B is a block diagram illustrating an example of a modeling unit according to the third example embodiment.

FIG. 11 is a block diagram illustrating an example of a prediction unit according to the third example embodiment.

FIG. 12 is a flowchart illustrating a processing example of the prediction system according to the third example embodiment.

FIG. 13 is a block diagram illustrating an example of a hardware configuration of an apparatus according to each example embodiment.

### EXAMPLE EMBODIMENTS

### First Example Embodiment

Hereinafter, a first example embodiment will be described with reference to the drawings. The first example embodiment discloses a prediction model generation apparatus according to the disclosed technology.

### [Description of Configuration]

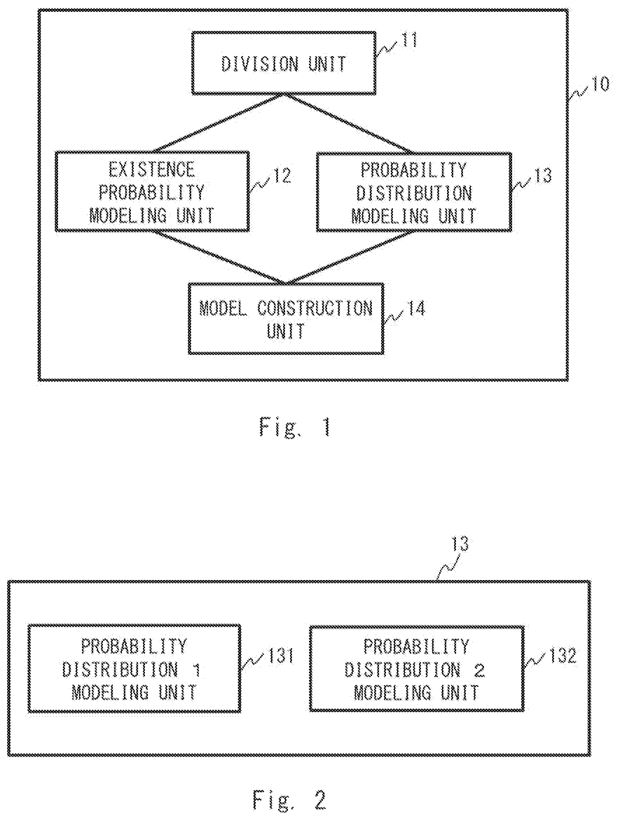

FIG. 1 illustrates an example of a prediction model generation apparatus according to the first example embodiment. The prediction model generation apparatus 10 in FIG. 1 includes a division unit 11, an existence probability modeling unit 12, a probability distribution modeling unit 13, and a model construction unit 14. Each unit (each means) of the prediction model generation apparatus 10 is controlled by a control unit (controller) (not illustrated). Each unit will be described below.

The division unit 11 divides a region for learning data including an objective variable where the probability distribution of the objective variable exists into a plurality of small regions according to the property of the objective variable. The property of the objective variable is that the probability that the objective variable becomes equal to or greater than a certain threshold value Th is significantly larger or smaller than the probability that the objective variable becomes less than the threshold value Th, for example. In this case, the division unit 11 divides the region where the probability distribution exists into two regions, that is, a region where the objective variable is equal to or greater than the threshold value Th and a region where the objective variable is less than the threshold value Th.

The threshold value Th in the above example may be a predetermined value or a value depending on an explanatory variable. For example, when a value of a certain explanatory variable is i, the division unit 11 can set a rule for the division method such as a region in which the objective variable is less than i as a small region 1 after the division and a region in which the objective variable is i or more as a small region 2. This is the setting of the division method where the explanatory variable takes on the initial value of the objective variable and the probability that the objective variable will take on a value equal to or greater than the explanatory variable is significantly high. The division unit 11 can set this rule for the division method by modeling through learning, for example. Based on this rule, the division unit 11 divides the region where the probability distribution of the objective variable exists.

However, the division unit 11 may divide the region into three or more divisions. For example, if a first probability that the objective variable becomes equal to or greater than a first threshold value Th1, a second probability that the objective variable becomes less than the first threshold value Th1 and greater than or equal to a second threshold value Th2 (Th1>Th2), and a third probability that the objective variable becomes less than the second threshold value Th2 are compared, it is conceivable that any one of the three probabilities is significantly larger than at least one of the other probabilities. In this case, the division unit 11 divides the region where the probability distribution of the objective variable exists into three small regions, that is, a small region where the objective variable is equal to or greater than the first threshold value Th1, a small region where the objective variable is less than the first threshold value Th1 and equal to or greater than the second threshold value Th2, and a small region where the objective variable is less than the second threshold value Th2.

The existence probability modeling unit 12 models, for a certain explanatory variable, the existence probability that the objective variable belongs to each of the small regions divided by the division unit 11. For example, if the division unit 11 divides the region into two, the existence probability modeling unit 12 derives the probability that the objective variable belongs to the small region 1 and the probability that the objective variable belongs to the small region 2 by modeling.

For a certain explanatory variable, the probability distribution modeling unit 13 uses learning data to model, for each of the divided small regions, a probability distribution related to a possible value of the objective variable in the small region under the condition that the objective variable belongs to the small region.

FIG. 2 is a block diagram illustrating an example of the probability distribution modeling unit 13. FIG. 2 illustrates an example in a case where there are two small regions, and the probability distribution modeling unit 13 includes a probability distribution 1 modeling unit 131 corresponding to the small region 1 and a probability distribution 2 modeling unit 132 corresponding to the small region 2. Under the condition that the objective variable belongs to the small region 1, the probability distribution 1 modeling unit 131 models a probability distribution related to a possible value of the objective variable in the small region 1. In this modeling, it is necessary to perform modeling such that the value of the probability distribution becomes 0 in a range in which the objective variable belongs to the small region 2.

Under the condition that the objective variable belongs to the small region 2, the probability distribution 2 modeling unit 132 models a probability distribution related to a possible value of the objective variable in the small region 2. In this modeling, it is necessary to perform modeling such that the value of the probability distribution becomes 0 in a range in which the objective variable belongs to the small region 1.

For a certain explanatory variable, the model construction unit 14 constructs a prediction model of the objective variable by integrating the probability distributions modeled by the probability distribution modeling unit 13 in small regions, using the existence probability modeled by the existence probability modeling unit 12. Specifically, the probability distributions are integrated using the fundamental law of probability (addition theorem and multiplication theorem).

As an example, in a case where a region is divided into two small regions, an objective variable is denoted as Y, an explanatory variable is denoted as X, and a variable indicating which of the two small regions the objective variable belongs to is denoted as Z. The model construction unit 14 derives a probability that the objective variable is Y under the condition that the explanatory variable is X, according to the following equation (1):

[Equation 1]

P(Y|X)=Σ_{Z}P(Y|X,Z)*P(Z|X) (1)

The left side of the equation (1) is a probability distribution of the objective variable Y to be derived. In the equation (1), P(Y|X, Z), which is the first term on the right side is a probability distribution of the objective variable under the condition that the explanatory variable is X and under the condition that the objective variable Y belongs to either the small region 1 or 2. In the equation (1), P(Z|X), which is the second term on the right side, gives a weight of combination of these two distributions, and is a probability indicating which of the small regions 1 and 2 the objective variable belongs to under the condition that the explanatory variable is X. Herein, P(Y|X, Z) and P(Z|X) are derived by the probability distribution modeling unit 13 and the existence probability modeling unit 12, respectively. However, description of variables (model parameters) used for modeling is omitted for simplicity.

### [Description of Processing]

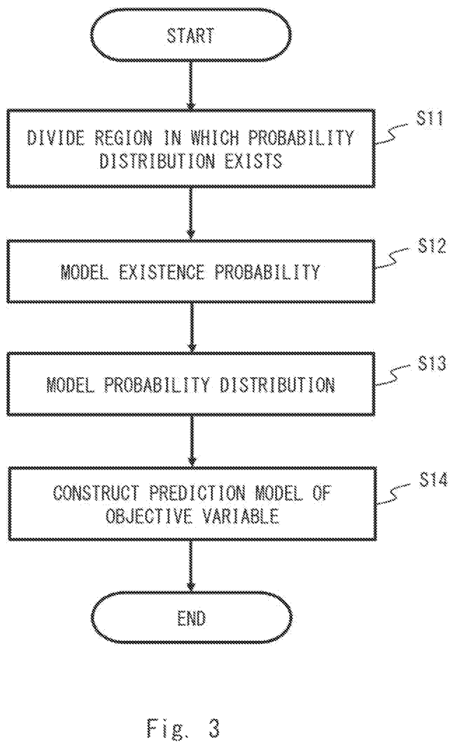

FIG. 3 is a flowchart illustrating an example of representative processing of the prediction model generation apparatus 10. The processing of the prediction model generation apparatus 10 will be described with reference to this flowchart. First, the division unit 11 of the prediction model generation apparatus divides a region where a probability distribution of an objective variable exists into a plurality of small regions according to the property of the objective variable for learning data including the objective variable (step S11: division step). The existence probability modeling unit 12 models an existence probability that the objective variable belongs to each of the divided small regions (step S12: existence probability modeling step).

The probability distribution modeling unit 13 uses learning data to model, for each of the small regions, a probability distribution related to a possible value of the objective variable in the small region under the condition that the objective variable belongs to the small region (step S13: probability distribution modeling step). The model construction unit 14 constructs a prediction model of the objective variable by integrating the modeled probability distributions in the small regions using the existence probability (step S14: model building step). Details of each step are as described above.

### DESCRIPTION OF ADVANTAGEOUS EFFECTS

As described above, the prediction model generation apparatus 10 can construct a prediction model of an objective variable for a certain explanatory variable. When there is a plurality of possible explanatory variables in the learning data, the prediction model generation apparatus 10 can construct the prediction model of the objective variable for each explanatory variable by executing the above-described processing for each possible explanatory variable.

Depending on the property of the objective variable, the probability distribution may be biased. In such a case, for example, if a prediction model is constructed from the learning data using a generalized linear model assuming a binomial distribution as it is, the property of the actual probability distribution is hardly reflected in the constructed prediction model, and the accuracy of prediction may be degraded.

However, in the prediction model generation apparatus 10 according to this disclosure, the division unit 11 divides a region where a probability distribution of an objective variable exists into a plurality of small regions according to the property of the objective variable, and the existence probability modeling unit 12 and the probability distribution modeling unit 13 execute modeling regarding the small regions. Then, the model construction unit 14 constructs a prediction model on the basis of the results derived by the existence probability modeling unit 12 and the probability distribution modeling unit 13. Therefore, the property of the actual probability distribution is likely to be reflected in the constructed prediction model, and the accuracy of prediction can be improved.

### Second Example Embodiment

Hereinafter, a second example embodiment will be described with reference to the drawings. The second example embodiment discloses a specific example of a prediction system having the function of the prediction model generation apparatus in the first example embodiment described above.

### [Description of Configuration]

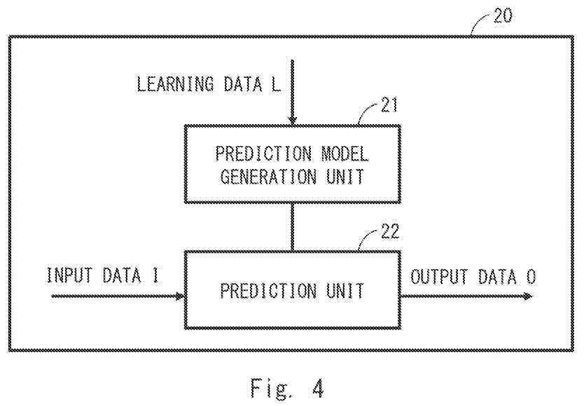

FIG. 4 illustrates an example of a prediction system according to the second example embodiment. A prediction system 20 in FIG. 4 includes a prediction model generation unit 21 and a prediction unit 22. The prediction model generation unit 21 is a unit that has the function of the prediction model generation apparatus 10 according to the first example embodiment and generates a prediction model using learning data L.

The learning data L is data for machine learning related to a plurality of patients, and has a degree of recovery upon hospitalization and patient information as explanatory variables for each of the plurality of patients, and has information on the degree of recovery at discharge from hospital as an objective variable corresponding to the explanatory variables. The degree of recovery takes on a quantified value indicating the degree of recovery from a predetermined disease, and is determined by a doctor or the like through an examination. Herein, the smaller the degree of recovery, the greater the disease state, and the larger the degree of recovery, the lighter the disease state. The degree of recovery upon hospitalization is an initial value of the degree of recovery at discharge from hospital. The patient information is information about a patient other than the degree of recovery upon hospitalization, which affects the degree of recovery at discharge from hospital. Examples of the patient information include, but are not limited to, age and a predetermined vital value of the patient's body.

New input data I (prediction target data) is information including a set of the degree of recovery upon hospitalization and patient information of a patient (prediction target patient) to be predicted by the prediction system 20. The prediction unit 22 selects one of the prediction models generated by the prediction model generation unit 21 and inputs the input data I to the prediction model as an explanatory variable, thereby to derive the degree of recovery at discharge from hospital of the prediction target patient that is the objective variable, as output data O.

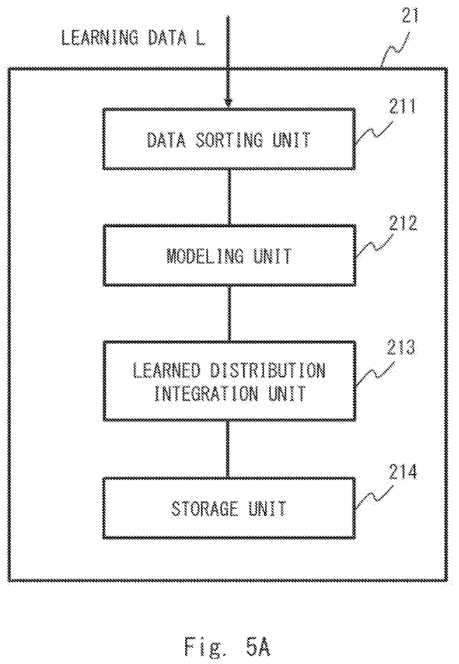

FIG. 5A is a block diagram illustrating an example of the prediction model generation unit 21. The prediction model generation unit 21 includes a data sorting unit 211, a modeling unit 212, a learned distribution integration unit 213, and a storage unit 214. Details of these units will be described below.

The data sorting unit 211 acquires the learning data L and sorts the learning data L on the basis of the value of the degree of recovery upon hospitalization that is the explanatory variable. In this example, since the degree of recovery can take N values of 1 to N, the learning data L is also divided into N pieces. The divided learning data is input to the modeling unit 212.

The modeling unit 212 constructs a prediction model of the degree of recovery at discharge from hospital (objective variable) for each value of the degree of recovery upon hospitalization. As illustrated in FIG. 5B, the modeling unit 212 includes N learning units 1 to N according to N values of the degrees of recovery upon hospitalization. The learning unit i (i is an arbitrary value of 1 to N) is distinguished by the value i of the degree of recovery upon hospitalization, and is labeled by the value of the degree of recovery upon hospitalization. Hereinafter, processing executed by the learning unit i will be described.

The learning unit i learns a probability distribution in a case where the degree of recovery upon hospitalization is i. Therefore, among the learning data L divided by the data sorting unit 211, the learning data L of the patient whose degree of recovery upon hospitalization is i is input to the learning unit i. The input learning data L includes, for each patient, patient information and the degree of recovery at discharge from hospital that is an objective variable.

The learning unit i includes a division unit 11, an existence probability modeling unit 12, and a probability distribution modeling unit 13 illustrated in FIG. 1. The learning unit i performs the following three types of learning: the learning of a parameter for a probability distribution of whether the degree of recovery at discharge from hospital is higher or lower than i, the learning of a parameter for a probability distribution of the degree of recovery at discharge from hospital under the condition that the degree of recovery at discharge from hospital is lower than i, and the learning of a parameter for a probability distribution of the degree of recovery at discharge from hospital under the condition that the degree of recovery at discharge from hospital is higher than i.

Herein, it is necessary to pay attention to a situation in which the degree of recovery upon hospitalization is the same as the degree of recovery at discharge from hospital. In the case of dividing the region where the probability distribution of the degree of recovery at discharge from hospital is present into two, this situation is included in the definition of whether the degree of recovery at discharge from hospital is higher or lower than the degree of recovery upon hospitalization. In this example, this situation is included in the former definition, and the case where the degree of recovery at discharge from hospital is equal to or higher than the degree of recovery upon hospitalization is redefined. In a case where the degree of recovery at discharge from hospital is lower than the degree of recovery upon hospitalization, it can be defined that the degree of recovery at discharge from hospital is less than the degree of recovery upon hospitalization. Therefore, the region where the probability distribution of the degree of recovery at discharge from hospital exists is divided into two, with the value of the degree of recovery upon hospitalization as a boundary. Then, the learning unit i models the distribution of the degree of recovery at discharge from hospital in each of the two conditions, that is, whether the degree of recovery at discharge from hospital is equal to or higher than or is lower than the degree of recovery upon hospitalization, using a binomial distribution.

This binomial distribution is characterized by two parameters: an integer parameter (hereinafter, described as the number of trials) and a real number parameter (hereinafter, described as success probability). Under the condition that the degree of recovery at discharge from hospital is less than the degree of recovery upon hospitalization, a value that can be taken as the degree of recovery at discharge from hospital is 1 to i−1, and thus, the learning unit i assumes a binomial distribution in which the number of trials is i−2. On the other hand, under the condition that the degree of recovery at discharge from hospital is equal to or greater than the degree of recovery upon hospitalization, a value that can be taken as the degree of recovery at discharge from hospital is i to N, and thus, the learning unit i assumes a binomial distribution in which the number of trials is N−i. In this way, the probability distribution that has been subjected to generalized linear modeling is modeled.

The learning unit i models the success probability of each probability distribution so as to depend on the patient information. As a link function used for modeling, a log it function is generally adopted. Processing such as point estimation or Bayesian estimation may be performed on the model parameters in learning.

From the above, each unit of the learning unit i executes the following processing. The division unit 11 divides a region in which the probability distribution of the degree of recovery at discharge from hospital exists into two regions: a small region 1 in which the degree of recovery at discharge from hospital is equal to or greater than the degree of recovery upon hospitalization and a small region 2 in which the degree of recovery at discharge from hospital is less than the degree of recovery upon hospitalization in accordance with the property of the degree of recovery at discharge from hospital (objective variable). The existence probability modeling unit 12 learns and models the existence probability that the degree of recovery at discharge from hospital belongs to each of the small regions 1 and 2 in the case where the degree of recovery upon hospitalization (explanatory variable) is i. Under the condition that the degree of recovery at discharge from hospital belongs to the small region 1, the probability distribution 1 modeling unit 131 learns and models a probability distribution related to a possible value (N to i) of the degree of recovery at discharge from hospital in the small region 1. The probability distribution 2 modeling unit 132 learns and models a probability distribution related to a possible value (1 to i−1) of the degree of recovery at discharge from hospital in the small region 2 under the condition that the degree of recovery at discharge from hospital belongs to the small region 2. As described above, each modeling unit uses the binomial distribution in the modeling.

The learned distribution integration unit 213 includes the model construction unit 14 illustrated in FIG. 1, and constructs a prediction model of the degree of recovery at discharge from hospital (objective variable) under the condition that the degree of recovery upon hospitalization (explanatory variable) is i. That is, according to the probability addition theorem and the multiplication theorem, the learned distribution integration unit 213 integrates three distributions, that is, the probability distribution indicating whether the degree of recovery at discharge from hospital is equal to or higher than or lower than the degree of recovery upon hospitalization, the probability distribution of the degree of recovery at discharge from hospital under the condition that the degree of recovery at discharge from hospital is equal to or higher than the degree of recovery upon hospitalization, and the probability distribution of the degree of recovery at discharge from hospital under the condition that the degree of recovery at discharge from hospital is lower than the degree of recovery upon hospitalization. Accordingly, the learned distribution integration unit 213 constructs a prediction model of the degree of recovery at discharge from hospital in a case where the degree of recovery upon hospitalization is i. The integration method is as described in the description of the equation (1).

The learned distribution integration unit 213 executes this process for all the values 1 to N that can be taken by the degree of recovery upon hospitalization. Accordingly, the learned distribution integration unit 213 constructs total N types of prediction models corresponding to the degrees of recovery upon hospitalization. The data sorting unit 211, the modeling unit 212, and the learned distribution integration unit 213 may execute the above processing related to machine learning each time the prediction model generation unit 21 acquires new learning data L. This makes it possible to update the prediction model and improve its accuracy.

The storage unit 214 stores the N types of prediction models constructed by the learned distribution integration unit 213. The N prediction models can be distinguished by being attached with identification information corresponding to the value of the degree of recovery upon hospitalization. The storage unit 214 also accepts access by a prediction model selection unit 221 described later.

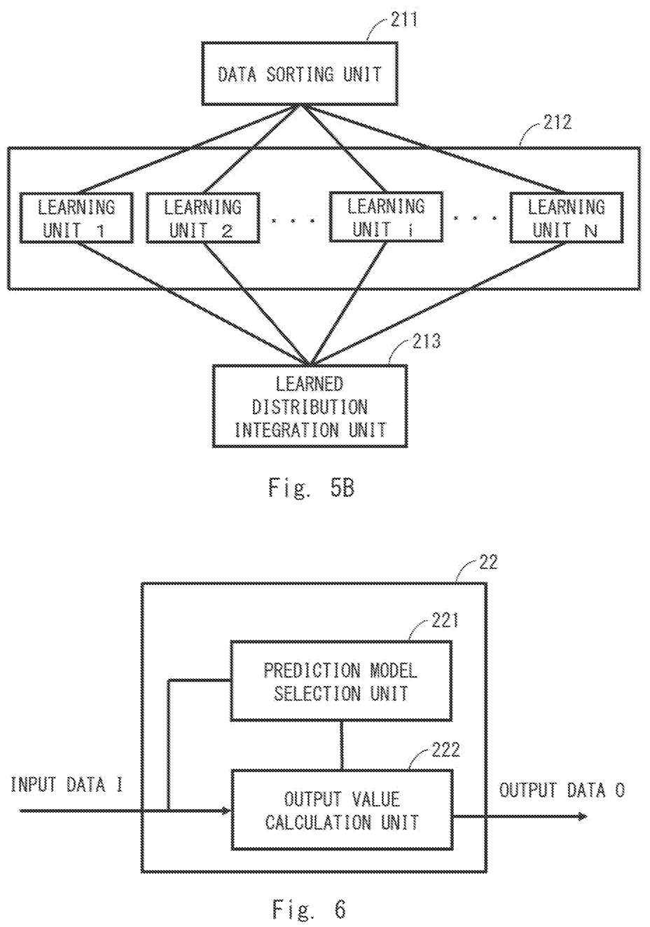

FIG. 6 is a block diagram illustrating an example of the prediction unit 22. The prediction unit 22 includes a prediction model selection unit 221 and an output value calculation unit 222. Details of these units will be described below.

The prediction model selection unit 221 accesses the storage unit 214 in accordance with the value of the degree of recovery upon hospitalization of the input data I, and selects one appropriate prediction model from among the N types of prediction models stored in the storage unit 214. For example, if the degree of recovery upon hospitalization of the input data I is 3, the prediction model selection unit 221 selects a prediction model in which the degree of recovery upon hospitalization is 3. The prediction model selection unit 221 can select a specific prediction model with reference to the identification information attached to the prediction model.

The output value calculation unit 222 inputs the patient information of the input data I to the prediction model selected by the prediction model selection unit 221, thereby acquiring a prediction distribution of the degree of recovery at discharge from hospital as an objective variable. The output value calculation unit 222 calculates any one of the mode value, the average value, and the median value of the prediction distribution, as a prediction value of the degree of recovery at discharge from hospital, and outputs this value as output data O. However, the method for calculating the prediction value is not limited to the above method. The output data O may be displayed on a display unit provided in the prediction system 20, or may be printed and output by a printer, for example.

### [Description of Processing]

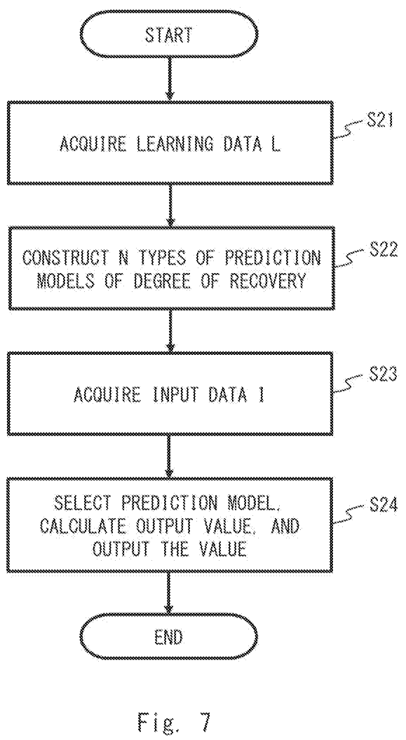

FIG. 7 is a flowchart illustrating an example of representative processing of the prediction system 20, and processing of the prediction system 20 will be described with this flowchart. First, the data sorting unit 211 in the prediction system 20 acquires the learning data L (step S21).

The data sorting unit 211 divides the acquired learning data L on the basis of the value of the degree of recovery upon hospitalization, and allocates the divided learning data L to the learning units 1 to N in the modeling unit 212. The learning unit i executes modeling under the condition that the degree of recovery upon hospitalization is i. Details of the modeling are as described above. Then, the learned distribution integration unit 213 constructs a prediction model of the degree of recovery at discharge from hospital under the condition that the degree of recovery upon hospitalization is i. The learned distribution integration unit 213 constructs total N types of prediction models by executing this processing even in a case where the degree of recovery upon hospitalization is a value other than i (step S22). The constructed prediction models are stored in the storage unit 214.

Next, the prediction unit 22 acquires the input data I (step S23). The prediction model selection unit 221 selects one learned prediction model according to the value of the degree of recovery upon hospitalization of the input data I. The output value calculation unit 222 acquires a predicted distribution of the degree of recovery at discharge from hospital by inputting the patient information of the input data I to the selected prediction model, and calculates a predicted value of the degree of recovery at discharge from hospital on the basis of the predicted distribution. The output value calculation unit 222 outputs the calculation results as output data O (step S24).

### DESCRIPTION OF ADVANTAGEOUS EFFECTS

As described above, the prediction system 20 can construct the prediction model of the degree of recovery of the patient with high accuracy using the learning data on the degree of recovery of the patient.

The learning data has the degree of recovery upon hospitalization (initial value of the objective variable) as an initial value of the degree of recovery at discharge from hospital (objective variable), and the prediction model generation unit 21 (model construction means) can construct a prediction model of the objective variable for each possible value of the degree of recovery upon hospitalization. Therefore, it is possible to predict the degree of recovery at discharge from hospital with respect to an arbitrary degree of recovery upon hospitalization.

When the input data I (prediction target data) having the degree of recovery upon hospitalization is input, the prediction model selection unit 221 (selection means) selects a prediction model corresponding to the degree of recovery upon hospitalization included in the input data I from among the constructed prediction models. The output value calculation unit 222 (prediction means) can predict the degree of recovery at discharge from hospital in the input data I using the selected prediction model. Therefore, the prediction system 20 can accurately predict the degree of recovery at discharge from hospital for the input data I of an arbitrary patient.

The objective variable in the learning data is the degree of recovery at discharge from hospital of the patient, and the data sorting unit 211 (division means) can divide the region in which the degree of recovery at discharge from hospital exists into two, with the value of the degree of recovery upon hospitalization of the patient as a boundary. Therefore, the prediction model can reflect an actual change in the degree of recovery from hospitalization to discharge from hospital. This point will be described in more detail in relation to a third example embodiment.

The learning data includes the patient information of the patient, and the learning unit i (probability distribution modeling means) can model a probability distribution so as to depend on the patient information. Therefore, the prediction model can reflect the patient information.

The learning unit i can also model the probability distribution that has been subjected to generalized linear modeling (in particular, a probability distribution represented by a binomial distribution). Therefore, the prediction system 20 can generate a highly accurate prediction model using a general method that is not special as a statistical method.

### Third Example Embodiment

Hereinafter, a third example embodiment will be described with reference to the drawings. In the third example embodiment, as a further specific example of the second example embodiment, a case where a functional independence measure (FIM) of a stroke patient is applied as the degree of recovery will be described.

For a recovery phase rehabilitation ward for stroke patients, individually predict the degree of recovery of each individual patient at discharge from hospital using patient information at the time of being hospitalized in the ward is important for planning a rehabilitation plan and setting a target of the patient. As an example, Non-Patent Literature 1 (“Prediction of Functional Independence Measure at Discharge using Patient Hospital Admission Data”, authors: Yuki Kosaka (NEC Data Science Research Laboratories), Toshinori Hosoi (NEC Data Science Research Laboratories), Masahiro Kubo (NEC Data Science Research Laboratories), Yoshikazu Kameda (KNI), Himeka Inoue (KNI), Akira Okuda (KNI), Ayaka Kubo (KNI), Miyuki Ito (KNI), material name: Collected Articles of the Conferences on Medical Informatics (CD-ROM), volume: 39th, page: ROMBUNNO. 3-B-2-03, year of publication: 2019) describes a regression method assuming FIM distribution at discharge as a Gaussian distribution to solve an issue of prediction of FIM at discharge.

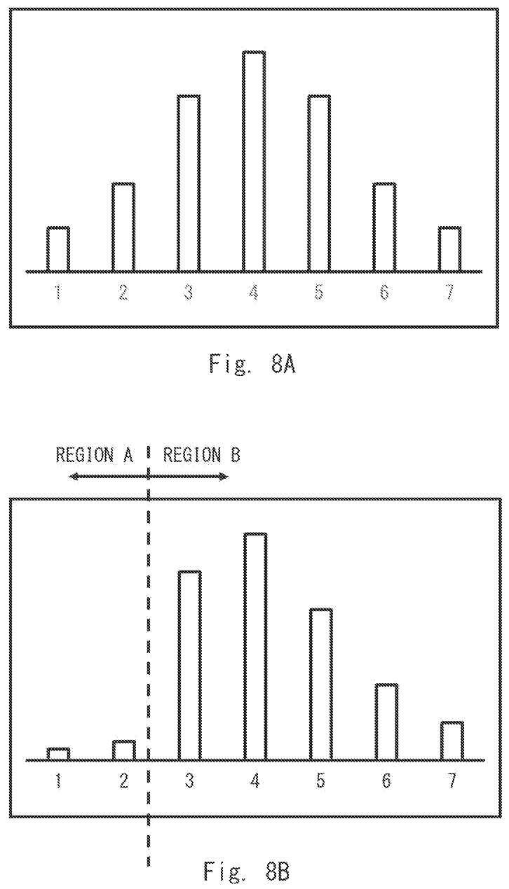

The quantity of the degree of recovery of a stroke patient represented by FIM or the like takes on a discrete value, which has an upper limit and a lower limit. Examples of a method of regressing a quantity having such a domain property include a generalized linear model assuming a binomial distribution. FIG. 8A illustrates an example of probability distribution of FIM in this model. The horizontal axis in FIG. 8A represents the FIM which takes on a value indicated by 1 to 7. That is, N in the second example embodiment is 7. The vertical axis represents the distribution intensity. With the intermediate value of 4 of the FIM as a boundary, the distribution intensity corresponding to the FIM decreases as the FIM increases or decreases. The manner of decrease in the distribution intensity is relatively gentle as illustrated in FIG. 8A.

However, the actual distribution of the FIM at discharge from hospital greatly will change between the first region and the second region with the value of the FIM upon hospitalization as a boundary, and it is predicted that there is a gap in the distribution between the two regions. FIG. 8B illustrates an example of the probability distribution of the FIM in such a model. In FIG. 8A, the horizontal axis represents the FIM (1 to 7), and the vertical axis represents the distribution intensity. In FIG. 8B, the FIM upon hospitalization is 3.

In FIG. 8B, while the distribution intensity of the region (region A) where the FIM at discharge from hospital is less than the FIM upon hospitalization is extremely small, the distribution intensity of the region (region B) where the FIM at discharge from hospital is equal to or more than the FIM upon hospitalization is extremely large. This is because an event that the FIM deteriorates due to rehabilitation during hospitalization rarely occurs. For the above reason, in the generalized linear model assuming the binomial distribution, such the property of the actual distribution cannot be reflected, and thus, there is a possibility that the accuracy of the FIM prediction is lowered.

A prediction system according to a third example embodiment described below can solve this problem. The prediction system according to the third example embodiment has substantially the same configuration as the second example embodiment, and thus differences from the second example embodiment will be particularly described, and description of other points will be omitted as appropriate.

### [Description of Configuration]

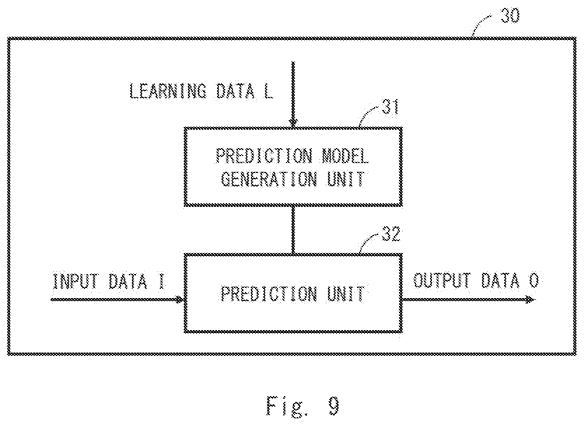

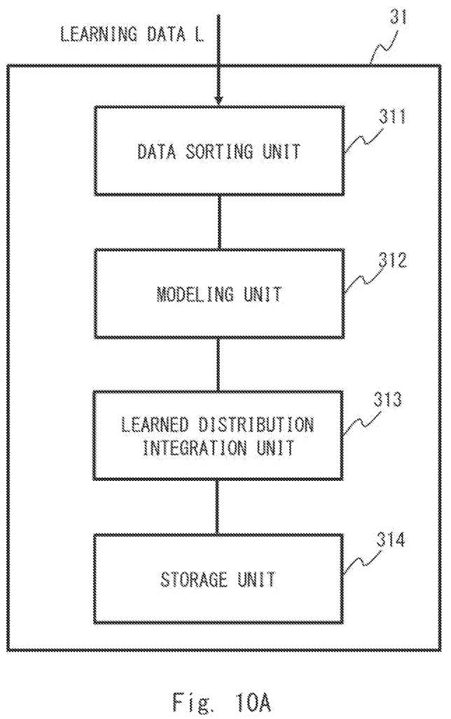

FIG. 9 illustrates an example of a prediction system according to the third example embodiment. A prediction system 30 includes a prediction model generation unit 31 and a prediction unit 32. The prediction model generation unit 31 and the prediction unit 32 correspond to the prediction model generation unit 21 and the prediction unit 22 in the second example embodiment, respectively.

The learning data L is data for machine learning related to a plurality of patients, and has an FIM upon hospitalization and patient information as explanatory variables for each of the plurality of patients, and has information on the FIM at discharge from hospital as an objective variable corresponding to the explanatory variables. The FIM is an example of the degree of recovery in the second example embodiment described above, and can take on a value of 1 to 7. The details of the patient information are as in the second example embodiment.

New input data I is information including a set of FIM upon hospitalization and patient information of a prediction target patient. The prediction unit 32 selects one prediction model generated by the prediction model generation unit 31 and inputs the input data I as an explanatory variable to the selected prediction model, thereby to derive the FIM at discharge from hospital that is the objective variable, as output data O.

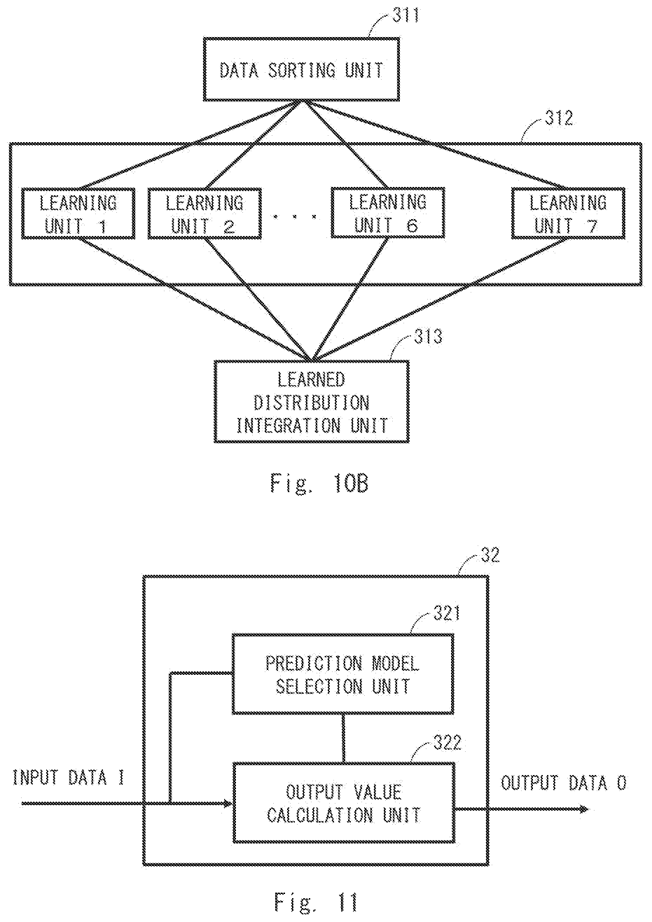

FIG. 10A is a block diagram illustrating an example of the prediction model generation unit 31. The prediction model generation unit 31 includes a data sorting unit 311, a modeling unit 312, a learned distribution integration unit 313, and a storage unit 314. The data sorting unit 311 to the storage unit 314 correspond to the data sorting unit 211 to the storage unit 214 in the second example embodiment, respectively.

The data sorting unit 311 acquires learning data L and divides the learning data L into seven pieces on the basis of the value of the FIM upon hospitalization. The modeling unit 312 constructs a prediction model of the FIM at discharge from hospital for each value of the FIM upon hospitalization. As illustrated in FIG. 10B, the modeling unit 312 includes seven learning units 1 to 7 according to the seven values of the FIM upon hospitalization. A learning unit i (i is an arbitrary value of 1 to 7) executes processing similar to that of the learning unit i described in the second example embodiment on the FIM instead of the degree of recovery. Under the condition that the FIM at discharge from hospital is equal to or greater than the FIM upon hospitalization, the value that can be taken as the FIM at discharge from hospital is i to 7, and thus, the learning unit i assumes a binomial distribution in which the number of trials is 7−i.

The learned distribution integration unit 313 integrates three probability distributions: a probability distribution indicating whether the FIM at discharge from hospital is equal to or greater than or less than the FIM upon hospitalization: a probability distribution of the FIM at discharge from hospital under a condition that the FIM at discharge from hospital is equal to or greater than the FIM upon hospitalization; and a probability distribution of the FIM at discharge from hospital under a condition that the FIM at discharge from hospital is less than the FIM upon hospitalization. Accordingly, the learned distribution integration unit 313 constructs a prediction model of the FIM at discharge from hospital in a case where the FIM upon hospitalization is i. The learned distribution integration unit 313 constructs total seven types of prediction models corresponding to the FIM upon hospitalization by executing this processing for all the values 1 to 7 that can be taken by the FIM upon hospitalization. The storage unit 314 stores the seven types of prediction models constructed by the learned distribution integration unit 313.

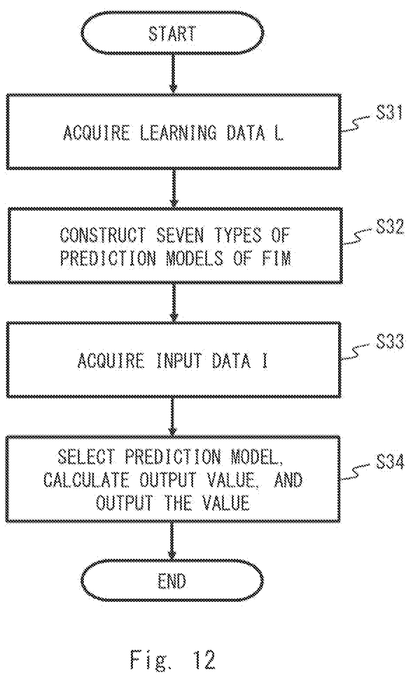

FIG. 11 is a block diagram illustrating an example of the prediction unit 32. The prediction unit 32 includes a prediction model selection unit 321 and an output value calculation unit 322. The prediction model selection unit 321 and the output value calculation unit 322 correspond to the prediction model selection unit 221 and the output value calculation unit 222 in the second example embodiment, respectively.

The prediction model selection unit 321 accesses the storage unit 314 in accordance with the value of the FIM upon hospitalization of the input data I, and selects one appropriate prediction model from among the seven types of prediction models stored in the storage unit 314. The output value calculation unit 322 acquires the predicted distribution of the FIM at discharge from hospital that is the objective variable by inputting the patient information of the input data I to the prediction model selected by the prediction model selection unit 321. The output value calculation unit 322 calculates a predicted value of the FIM at discharge from hospital on the basis of the predicted distribution.

### [Description of Processing]

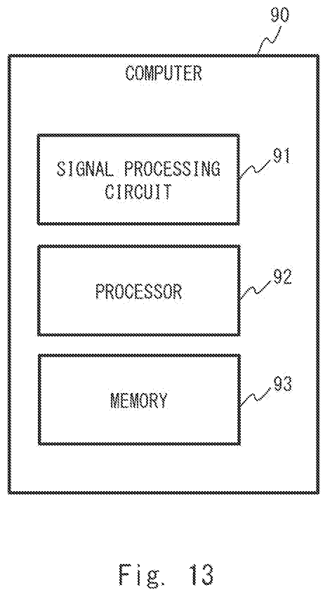

FIG. 12 is a flowchart illustrating an example of representative processing of the prediction system 30, and processing of the prediction system 30 will be described with this flowchart. First, the data sorting unit 311 of the prediction system 30 acquires the learning data L (step S31).

The data sorting unit 311 divides the acquired learning data L on the basis of the value of the FIM upon hospitalization, and allocates the divided learning data L to the learning units 1 to 7 of the modeling unit 312. The learning unit i executes modeling under the condition that the FIM upon hospitalization is i. Details of the modeling are as described above. Then, the learned distribution integration unit 313 constructs a prediction model of the FIM at discharge from hospital under the condition that the FIM upon hospitalization is i. The learned distribution integration unit 313 constructs total seven types of prediction models by executing this processing even in a case where the FIM upon hospitalization is a value other than i (step S32). The constructed prediction models are stored in the storage unit 314.

Next, the prediction unit 32 acquires the input data I (step S33). The prediction model selection unit 321 selects one learned prediction model according to the value of the FIM upon hospitalization of the input data I. The output value calculation unit 222 acquires a predicted distribution of the FIM at discharge from hospital by inputting the patient information of the input data I to the selected prediction model, and calculates a predicted value of the FIM at discharge from hospital on the basis of the predicted distribution. The output value calculation unit 222 outputs the calculation results as output data O (step S34).

### DESCRIPTION OF ADVANTAGEOUS EFFECTS

As described above, the prediction system 30 can construct the prediction model of the FIM of the patient with high accuracy using the learning data on the FIM of the patient. The prediction system 30 performs modeling of distribution in two regions, that is, a region where the FIM at discharge from hospital is equal to or higher than the FIM upon hospitalization and the other region. The prediction system 30 then models probability distributions under the condition that the probability distributions belong to either of the regions and integrates the calculated probability distributions to construct a prediction model. Accordingly, the constructed prediction model can approximate the shape of the actual distribution well, so that improvement in the prediction accuracy can be expected.

### Fourth Example Embodiment

Hereinafter, a fourth example embodiment will be described. In the fourth example embodiment, as a further specific example of the second example embodiment, a case where stroke impairment assessment set (SIAS) of a stroke patient is applied as the degree of recovery will be described. Regarding SIAS, for the same reason as FIM, there are very many cases where SIAS at discharge from hospital takes on a value equal to or more than SIAS upon hospitalization. Therefore, it is effective to apply the prediction system according to this disclosure.

The processing according to the fourth example embodiment can be implemented by replacing FIM in the third example embodiment (FIM prediction) with SIAS. However, since there are six or four possible values of SIAS, the value of N in the second example embodiment is 6 or 4 in the third example embodiment.

### Fifth Example Embodiment

Hereinafter, a fifth example embodiment will be described. In the fifth example embodiment, as a further specific example of the second example embodiment, a case where a Berg balance scale (BBS) which is evaluation of the balance function is applied to the degree of recovery will be described.

Regarding BBS, for the same reason as FIM, there are very many cases where BBS at discharge from hospital takes on a value equal to or more than BBS upon hospitalization. Therefore, it is effective to apply the prediction system according to this disclosure.

The processing according to the fifth example embodiment can be implemented by replacing FIM in the third example embodiment (FIM prediction) with BBS. However, since there are four possible values of BBS, the value of N in the second example embodiment is 4 in the third example embodiment.

As described above in the third to fifth example embodiments, the prediction system according to the present disclosure can be applied to prediction of various types of degrees of recovery.

Note that the present disclosure is not limited to the above-described example embodiments, and can be appropriately modified without departing from the gist.

For example, the output value calculation unit 222 according to the second example embodiment may output the prediction distribution of the degree of recovery at discharge from hospital as it is, or may calculate a possible value of the degree of recovery at discharge from hospital and a probability of the value based on the prediction distribution and output the calculated information. In the second example embodiment, a situation in which the degree of recovery upon hospitalization is the same as the degree of recovery at discharge from hospital may be included in a definition in which the degree of recovery at discharge from hospital is lower than the degree of recovery upon hospitalization. The boundary in the division is not limited to the same value as the value of the degree of recovery upon hospitalization, and may take on a different value. Similar modifications are possible not only in the second example embodiment but also in the third to fifth example embodiments.

The prediction model generation apparatus 10 according to the first example embodiment may have a centralized configuration including a single computer, or may have a distributed configuration in which a plurality of computers shares and executes the processing of the division unit 11 to the model construction unit 14. Similarly, the prediction systems according to the second to fifth example embodiments may have a centralized configuration including a single computer, or may have a distributed configuration in which a plurality of computers shares and executes the processing. For example, the prediction system 20 may be configured such that a first computer includes the prediction model generation unit 21 and executes the processing, and a second computer includes the prediction unit 22 and executes the processing. In the distributed configuration, the plurality of devices may be connected via a communication network such as a local area network (LAN), a wide area network (WAN), or the Internet.

The prediction model generation apparatus or the prediction system according to the present disclosure can be widely applied not only to the degree of recovery but also to the purpose for predicting a future value of an amount (variable) whose initial value is known. In particular, it is effective in the prediction of a phenomenon in which the increase or decrease from the initial value with a lapse of time is biased to either one. For example, the prediction model generation apparatus or the prediction system according to the present disclosure can also be applied to the purpose for predicting a future value of an amount of a capability that clearly tends to decrease with aging, such as hearing, vision, or the like. In this case, the current value of hearing or vision is treated as an initial value, and the future value of hearing or vision is an objective variable to be predicted.

In the example embodiments described above, the present disclosure has been described as a hardware configuration, but the present disclosure is not limited thereto. In the present disclosure, the processing (steps) in the prediction model generation apparatus or the prediction system described in the above-described example embodiments can be also implemented by causing a processor in a computer to execute a computer program.

FIG. 13 is a block diagram illustrating a hardware configuration example of an information processing device (signal processing device) in which the processes in each example embodiment described above are executed. Referring to FIG. 13, an information processing apparatus 90 includes a signal processing circuit 91, a processor 92, and a memory 93.

The signal processing circuit 91 is a circuit for processing a signal under the control of the processor 92. The signal processing circuit 91 may include a communication circuit that receives a signal from a signal transmission apparatus.

The processor 92 reads and executes software (computer program) from the memory 93 to execute the processing in the apparatus described in the above-described example embodiments. As an example of the processor 92, one of a central processing unit (CPU), a micro processing unit (MPU), a field-programmable gate array (FPGA), a demand-side platform (DSP), or an application specific integrated circuit (ASIC) may be used, or a plurality of processors may be used in combination.

The memory 93 includes a volatile memory, a nonvolatile memory, or a combination thereof. The number of memories 93 is not limited to one, and a plurality of memories may be provided. The volatile memory may be, for example, a random access memory (RAM) such as a dynamic random access memory (DRAM) or a static random access memory (SRAM). The nonvolatile memory may be, for example, a random only memory (ROM) such as a programmable random only memory (PROM) or an erasable programmable read only memory (EPROM), or a solid state drive (SSD).

The memory 93 is used to store one or more instructions. Here, one or more instructions are stored in the memory 93 as a software module group. The processor 92 can execute the processes described in the above example embodiments by reading and executing the software module group from the memory 93.

Note that the memory 93 may include a memory built in the processor 92 in addition to a memory provided outside the processor 92. The memory 93 may include a storage disposed away from a processor configuring the processor 92. In this case, the processor 92 can access the memory 93 via an input/output (I/O) interface.

As described above, one or a plurality of processors included in each apparatus in the above example embodiments execute one or a plurality of programs including an instruction group for causing a computer to execute an algorithm described with reference to the drawings. With the above processes, the signal processing method described in each example embodiment can be realized.

The program may be stored by using various types of non-transitory computer readable media to be supplied to a computer. The non-transitory computer readable media include various types of tangible storage media. Examples of the non-transitory computer-readable medium include a magnetic recording medium (for example, a flexible disk, a magnetic tape, or a hard disk drive), an optical magnetic recording medium (for example, a magneto-optical disk), a CD-Read Only Memory (ROM), a CD-R, a CD-R/W, and a semiconductor memory (for example, a mask ROM, a programmable ROM (PROM), an erasable PROM (EPROM), a flash ROM, or a random access memory (RAM)). The program may be supplied to the computer by various types of transitory computer-readable media. Examples of the transitory computer-readable media include electrical signals, optical signals, and electromagnetic waves. The transitory computer-readable media can supply programs to computers via wired communication paths, such as wires and optical fiber, or wireless communication paths.

Although the present disclosure has been described above with reference to the example embodiments, the present disclosure is not limited to the above. Various modifications that could be understood by those skilled in the art can be made to the configuration and details of the present disclosure within the scope of the present disclosure.

### REFERENCE SIGNS LIST

10 PREDICTION MODEL GENERATION APPARATUS
11 DIVISION UNIT
12 EXISTENCE PROBABILITY MODELING UNIT
13 PROBABILITY DISTRIBUTION MODELING UNIT
14 MODEL CONSTRUCTION UNIT
20 PREDICTION SYSTEM
21 PREDICTION MODEL GENERATION UNIT
211 DATA SORTING UNIT
212 MODELING UNIT
213 LEARNED DISTRIBUTION INTEGRATION UNIT
214 STORAGE UNIT
22 PREDICTION UNIT
221 PREDICTION MODEL SELECTION UNIT
222 OUTPUT VALUE CALCULATION UNIT
30 PREDICTION SYSTEM
31 PREDICTION MODEL GENERATION UNIT
311 DATA SORTING UNIT
312 MODELING UNIT
313 LEARNED DISTRIBUTION INTEGRATION UNIT
314 STORAGE UNIT
32 PREDICTION UNIT
321 PREDICTION MODEL SELECTION UNIT
322 OUTPUT VALUE CALCULATION UNIT

What is claimed is:

1. A prediction model generation apparatus comprising:
at least one memory storing instructions; and
at least one processor configured to execute the instructions to:
divide a region in which a probability distribution of an objective variable exists into a plurality of small regions according to a property of the objective variable for learning data including the objective variable;
model an existence probability that the objective variable belongs to each of the small regions;
use the learning data to model, for each of the small regions, a probability distribution related to a possible value of the objective variable in the small region under a condition that the objective variable belongs to the small region; and
construct a prediction model of the objective variable by integrating the modeled probability distribution for each of the small regions using the existence probability.

2. The prediction model generation apparatus according to claim 1, wherein the learning data has an initial value of the objective variable, and the at least one processor is further configured to:
construct a prediction model of the objective variable for each possible value of the initial value.

3. The prediction model generation apparatus according to claim 2, wherein the at least one processor is further configured to:
select, when prediction target data having an initial value of an objective variable to be predicted is input, a prediction model corresponding to the initial value of the objective variable included in the prediction target data from among the prediction models of the constructed objective variable; and
predict the objective variable in the prediction target data using the selected prediction model.

4. The prediction model generation apparatus according to claim 1, wherein the objective variable is a degree of recovery of a patient at discharge from hospital, and the at least one processor is further configured to:
divide a region in which the degree of recovery at discharge from hospital exists into two, by using a value of the degree of recovery upon hospitalization of the patient as a boundary.

5. The prediction model generation apparatus according to claim 4, wherein the learning data includes patient information of the patient, and the at least one processor is further configured to:
model the probability distribution so as to depend on the patient information.

6. The prediction model generation apparatus according to claim 1, wherein the at least one processor is further configured to:
model the probability distribution that has been subjected to generalized linear modeling.

7. The prediction model generation apparatus according to claim 6, wherein the at least one processor is further configured to:
model the probability distribution represented by a binomial distribution.

8. A prediction model generation method executed by a prediction model generation apparatus, the method comprising:
dividing a region in which a probability distribution of an objective variable exists into a plurality of small regions according to a property of the objective variable for learning data including the objective variable;
modeling an existence probability that the objective variable belongs to each of the small regions;
using the learning data to model, for each of the small regions, a probability distribution related to a possible value of the objective variable in the small region under a condition that the objective variable belongs to the small region; and
constructing a prediction model of the objective variable by integrating the modeled probability distribution for each of the small regions using the existence probability.

9. A non-transitory computer-readable medium storing a program for causing a computer to perform:
dividing a region in which a probability distribution of an objective variable exists into a plurality of small regions according to a property of the objective variable for learning data including the objective variable;
modeling an existence probability that the objective variable belongs to each of the small regions;
using the learning data to model, for each of the small regions, a probability distribution related to a possible value of the objective variable in the small region under a condition that the objective variable belongs to the small region; and
constructing a prediction model of the objective variable by integrating the modeled probability distribution for each of the small regions using the existence probability.
